(19) United States
(12) Patent Application Publication
(10) Pub. No.: US 2019/0350867 A1
(43) Pub. Date: Nov. 21, 2019

(54) PREVENTION OF LOCAL TUMOR RECURRENCE FOLLOWING SURGERY USING SUSTAINED AND/OR DELAYED RELEASE OF MEDICAMENTS CONTAINED IN MICRO-PARTICLES

(71) Applicant: Soon Kap Hahn, Irvine, CA (US)

(72) Inventor: Soon Kap Hahn, Irvine, CA (US)

(21) Appl. No.: 15/854,225

(22) Filed: Dec. 26, 2017

Related U.S. Application Data
(60) Provisional application No. 62/439,429, filed on Dec. 27, 2016.

(51) Int. Cl. A61K 9/50

(52) U.S. Cl. CPC A61K 9/5031

(57) ABSTRACT

A compound and a method of use with a therapeutic medicament and a drug delivery system. The therapeutic medicament and the drug delivery system comprises two different types of micro-particles based on a biodegradable polyester whereby a first plurality of micro-particles (I) containing anticancer drug or medicament without an initial burst release and a second plurality of micro-particles (II) that releases wound healing drug or medicament.

### FIELD OF THE INVENTION

The present invention comprises a treatment process following cancer surgery. More specifically, the present invention comprises a local chemotherapy treatment process after surgery to prevent local cancer recurrence using a sustained, controlled chemotherapeutic or biologic therapy drug delivery system.

### BACKGROUND OF THE INVENTION

There are four major types of cancer treatment: 1) surgery 2) chemotherapy 3) radiation 4) biologic therapy. If the tumor appears to be confined to one area (localized and not metastatic), surgery may be used to remove it along with any nearby tissue that might contain cancerous cells. It is often difficult to pre-determine how large the surgerical resection will be. The surgeon generally makes that determination when (s)he sees the extent of the cancer during the operational procedure. Surgery is most successful when the cancer has not metastasized (spread to other organs). Surgery offers the greatest chance of cure or long-term remission for many types of cancer. However, even after surgery, cancer may recur after several years. This cancer recurrence may be due to some residual cancer cells which were not removed during the surgery. To kill these residual cancer cells, post-surgical treatments such as radiation therapy and/or systemic chemotherapy are performed. Darby et al. reported that post-surgical radiation therapy reduced the risk of recurrence during the 10 years after breast cancer surgery from 35% to 19.3% and reduced the risk of death from breast cancer from 25.2% to 21.4% over the first 15 years (Lancet, 1707-1716 (2011)). Currently many oncologists in the U.S. use the radiation therapy after lumpectomy (breast-conserving surgery) as standard procedure. However, it is expensive ($6,000-12,000) and cumbersome (4-5 weeks treatment). In addition, there are side effects, including skin burns and fatigue. Aebi et al. reported that systemic chemotherapy after surgery led to higher rates of disease-free and overall survival for women with isolated or regional recurrence of breast cancer (2012 CTRC-AACR San Antonio Breast Cancer Symposium). However, systemic chemotherapy is expensive and not successful in all treated patients. Studies in mice show that nearly 50% of systemically administered paclitaxel is excreted in the first day and less than 0.5% of the total dose remains locally available to treat cancer within the lung (Sparreboom et al. Anticancer Drugs, 78-86 (1996)). To reach effective levels locally by systemic delivery, high doses are necessary, which may lead to increased risk of systemic toxicity and morbidity.

Sustained and delayed release over several weeks of anticancer drug from a release form administered after surgery would enhance their efficacy in killing residual cancer cells without causing side effects or toxicity associated with the radiation therapy or systemic chemotherapy. Sustained release form of anticancer drug locally over several weeks would facilitate and ensure the killing of residual cancer cells at different cell cycle stages. One problem with immediately releasing an anticancer drug is that they are generally anti-proliferative and may inhibit wound healing following cancer surgery. The healing of wounds, including the wound caused by surgical resection, is a complex process that involves the activation and synchronization of many physiological events, including coagulatory and inflammatory events, fibrous tissue accretion, deposition of collagen, epithelialization, wound contraction, tissue granulation and remodeling. Disruptions caused by the addition of anti-proliferative drug such as anti-cancer drug can lead to chronic wounds that are difficult to manage. Most of sustained release drug delivery systems show initial burst release within the first 24-48 hours followed by sustained, uniform slow release of the encapsulated drug. This initial burst release of anti-cancer drug may impair wound healing. An ideal anticancer sustained, controlled release drug delivery system should possess the following properties:

1. Sustained and controlled release of anticancer drug over 12 weeks

2. Reduced 24-48-hour initial burst of anticancer drug

3. Acceleration of or not inhibiting wound healing during the first 7-10 days

4. Biodegradable, eliminating the need for surgical removal

A local anticancer drug delivery system possessing the above properties was developed and described by Colson et al. (Annals of Surgical Oncology, 1203-1213 (2010); U.S. Pat. Nos. 7,671,095; 8,334,324; 8,338,492, 8,795,707, Publication: US 2013/0195954 and 2014/0271489). This system used a film or micro-particle form which consists of poly(glycerol monostearate-co-ε-caprolactone) and paclitaxel. The paclitaxel-loaded polymer film was implanted on the dorsum of mice after the removal of primary cancer. The paclitaxel-loaded film prevented local cancer recurrence in 83.3% of mice, compared with 12.5% of unloaded film. These inventions claim that their polymer film avoids the initial burst release of paclitaxel by functionalization of the hydroxyl group in glycerol with hydrophobic stearic acid. However, the polymer developed and used in their system is difficult to synthesize and thus possesses less commercial value. In addition, the polymer is a novel form. Regulatory authorities would require proof of safety before approving it for use in medical applications. Brem et al. (US 2012/0121510 A1) described two methods to achieve the above ideal properties; 1) designing and preparing particles which do not release anticancer drugs for approximately 2-3 weeks and administering them immediately after surgery or 2) not administering particles which release anticancer drug immediately until 2-3 weeks after surgery. Method 1) is difficult to achieve at a commercial manufacturing scale; the authors did not clearly describe how to attain such particles. Method 2) requires injection by a syringe which will be painful to patients. In addition, the injection may not distribute particles in the entire area where there may be residual tumor cells. Ogura et al. (Surgery, 66-71 (2006)) applied a mixture of gemcitabine (anticancer drug) and fibrin glue (biocompatible hemostat) to the tail of the pancreas of nude mice which were injected with SUIT-2 human pancreatic cells. This local drug delivery system aimed to demonstrate the inhibition of proliferation of residual pancreatic cancer cells. GLIADEL® wafer is the most well-known local anticancer drug delivery system manufactured and commercialized by MGI Pharma. The GLIADEL® wafer made of a biodegradable polyanhydride delivers an anticancer drug (carmustine), when placed close to the resection margins, for treating malignant glioblastoma patients after surgery. It improves the survival rate of treated patients modestly and the quality of life compared to systemic chemotherapy. However, this drug delivery system does not consider wound healing of the resected area. Therefore, there is still a need for a better drug delivery system to reduce the likelihood of cancer recurrence without inhibiting the wound healing.

### SUMMARY OF THE INVENTION

The present invention consists of two different types of micro-particle based on biodegradable polyester:

1) a first plurality of micro-particle (I) containing anticancer drug without the initial burst release; and

2) a second plurality of micro-particle (II) releasing wound healing drugs.

The first plurality of micro-particle (I) releases anticancer drug over 12 weeks without an initial burst release. The second plurality of micro-particle (II) releases wound healing drugs within 7-10 days to accelerate wound healing. In a preferred embodiment, the present invention utilizes polylactic glycolic acid copolymer (PLGA) containing an anticancer drug such as paclitaxel for the first plurality of micro-particle (I), and wound healing drugs such as borneol and bismuth subgallate for the second plurality of micro-particle (II). All of the components in the preferred embodiment including PLGA, paclitaxel, borneol and bismuth subgallate are accepted and approved by the U.S. FDA and many other regulatory authorities worldwide. The two micro-particles (I) and (II) in this present invention can be mixed before applying to the entire area from which the tumor was removed to deliver a predetermined amount of anticancer drug and wound healing drugs before the surgical wound is closed.

### DESCRIPTION OF THE PREFERRED EMBODIMENTS

### Micro-Particle

Micro-particles used herein refer to particles having sizes between 1 μm and 500 μm and include microcapsules, microspheres and other particles. Micro-particles composed of drugs or medicaments and polymers are commonly used as a sustained, controlled release drug delivery system. Microcapsules generally have a drug core coated with a polymer film and may be spherical or non-spherical in shape. In contrast, microspheres have drugs dispersed evenly in polymer and are spherical in shape.

### Polymer

Micro-particles in this invention consist of biodegradable polymer and anticancer drug or wound healing drugs/medicament. Biodegradable polymers are defined as polymers that are degradable in vivo, either enzymatically or non-enzymatically, to produce non-toxic by-products. Biodegradable polymers have become increasingly important in pharmaceutical industry especially in the field of drug delivery. Biodegradable polymers can be formulated with drugs to form a drug delivery system which can provide sustained and controlled release of drugs over days, weeks or months. Since the drug delivery system based on biodegradable polymers degrades completely over time, it is not necessary to remove it by a surgical procedure after implanting or administration. Biodegradable polymers can be classified into natural biodegradable polymers or synthetic biodegradable polymers depending on their sources. Natural biodegradable polymers include gelatin, albumin, collagen, alginate, chitosan, derivatized cellulose, starch, hyaluronic acid and dextran. Synthetic biodegradable polymers include polyesters, polyurethanes, polyphosphazines, polyanhydrides, polycarbonates and polyesteramide. Polyesters include polylactic (PLA), polyglycolic (PGA), polycaprolactone (PCL) and their copolymers including well-known polylactic glycolic acid (PLGA). Their safety and usefulness as a drug delivery system are well-studied and accepted by regulatory authorities worldwide including the U.S. FDA. In one embodiment, the present invention uses polyesters. In a preferred embodiment, the present invention uses PLGA, a copolymer of PLA and PGA. Polyesters overall possess ideal physical and chemical properties providing ease to process, optimum drug release profile over days, weeks or months and non-toxic by-products after degradation.

### DRUGS or MEDICAMENTS

Anticancer drugs can be classified into chemotherapeutic drugs and biological drugs. Chemotherapeutic drugs can be further classified by their mode of action:

1) alkylating;

2) antimetabolite;

3) antimicrotuble;

4) topoisomerase inhibition; and

5) cytotoxic antibiotic.

Alkylating agents include nitrogen mustards, nitrosoureas, tetrazines, aziridines, cisplatin and its derivatives, and non-classical akylating agents such as procarbazine and hexamethylmelamine. Antimetabolites include anti-folates, fluoropyrimidines, deoxynuceloside analogues and thiopurines. Antimicrotubule agents include vinca alkaloids and taxanes including paclitaxel.

Topoisomerase inhibitors include irinotecan, topotecan and other analogues. Cytotoxic antibiotics include doxorubicin, daunorubicin, bleomycin and other analogues. Most of biological anticancer drugs try to enhance the patient's natural immune responses against cancer cells.

Currently monoclonal antibodies, interleukins and interferons are types of biological anti-cancer drug commonly used to treat various cancers. Monoclonal antibody-based biological anticancer drugs include Herceptin®, Rituxan®, Avastin® and other agents. As a new class of anti-cancer drugs, monoclonal antibodies can be conjugated with chemotherapeutic drugs. Monoclonal antibodies can be designed to target cancer cells specifically. The monoclonal antibodies conjugated with chemotherapeutic drugs can take the conjugated chemotherapeutic drugs and deliver them specifically to the cancer cells but not to other cells. This limits the damage to normal cells. Examples of these types of antibodies include brentuximab vedotin (Adcetris®) and ado-trastuzumab emtansine (Kadcyla®). The current invention uses any one of the above drugs or a combination of several drugs. In a preferred embodiment, anticancer drug encapsulated in micro-particle (I) is paclitaxel.

Wound healing drugs can be classified into small molecule drugs such as monoterpene-based or monoterpenoid-based drugs and biological drugs such as platelet-derived growth factor (PDGF) or other growth factors. The monoterpene-based or monoterpenoid-based drugs include borneol, thymol, genipin, α-terpineol and aucubin. To enhance wound healing, other synergistic component such as bismuth subgallate can be combined with the monoterpene-based drug. Sulbogin® consists of borneol and bismuth subgallate and is a wound healing product approved by the U.S. FDA in 2004 as an ointment form (U.S. Pat. No. 6,232,341). In a preferred embodiment, the present invention uses a combination of borneol and bismuth subgallate as wound healing drugs.

### Excipients

Micro-particles in the present invention may also contain one or more pharmaceutically acceptable additives. The term “additive” is all components contained in micro-particles other than drugs or polymer and includes, but not limited to, buffers, preservatives and antimicrobials. It can also include hydrophilic materials such as polyethylene glycol (PEG) or polyvinylpyrrolidone (PVP) which can accelerate the biodegradation of micro-particles.

### Micro-Particle Fabrication

Micro-particles in the present invention can be prepared by microencapsulation, spray drying, precipitation, hot melt microencapsulation, co-extrusion, precision particle fabrication (PPF) or other fabrication techniques. Microencapsulation techniques use single, double or multiple emulsion process in combination with solvent removal step such as evaporation, extraction or coacervation step. They are the most commonly used techniques to prepare micro-particles. The above techniques including the microencapsulation techniques can be used for water soluble drug, organic solvent soluble drug and solid powder drug. Polyesters can be processed with any one of the above techniques.

### Micro-Particle (I)

Micro-particle (I) consists of polyester and anticancer drug and can be prepared with any one of the techniques described in the previous section. Micro-particle (I) degrades slower and thus releases the contained drug longer than micro-particle (II). In a preferred embodiment, the present invention uses PLGA. Drug release rate from PLGA micro-particles can be controlled by adjusting a number of parameters such as 1) ratio between polylactic acid (PLA) and polyglycolic acid (PGA), 2) molecular weight and 3) size of micro-particle. In PLGA, polylactic acid is more hydrophobic compared to polyglycolic acid and subsequently hydrolyzes (i.e., degrades) slower. For example, PLGA 50:50 (PLA:PGA) exhibits a faster degradation than PLGA 75:25 due to preferential degradation of glycolic acid proportion if two polymers have the same molecular weights. PLGA with higher molecular weight exhibits a slower degradation rate than PLGA with lower molecular weight. Molecular weight has a direct relationship with the polymer chain size. Higher molecular weight PLGA has longer polymer chain and requires more time to degrade than lower molecular weight PLGA. In addition, an increase in molecular weight decreases drug diffusion rate and therefore drug release rate. The size of micro-particle also affects the rate of drug release. As the size of micro-particle decreases, the ratio of surface area to volume of the micro-particle increases. Thus, for a given rate of drug diffusion, the rate of drug release from the micro-particle will increase with decreasing micro-particle size. In addition, water penetration into smaller micro-particle may be quicker due to the shorter distance from the surface to the center of the micro-particle.

The delivered PLGA micro-particle (I) degrades and releases anticancer drug over 12 weeks to the cancer removed area and kill any residual cancer cells. Most chemotherapeutic drugs work by impairing mitosis (cell division). Most of cancer cells exhibit cell cycles of several days to several weeks. Therefore, effective drug delivery systems should release anticancer drugs over a long period (>4 weeks) to avoid the escape of cancer cells, which may become a source of cancer recurrence. Micro-particle (I) in the present invention can be prepared with PLGA 1) a portion of PLA equal to or greater than 50%, 2) molecular weight (Mw)>35,000 and 3) micro-particle size larger than 1 μm. Preferably the size of micro-particle (I) is larger than 50 μm. Typical composition of micro-particles (I) in this invention is 60-95% of PLGA and 5-40% of anticancer drug. In a preferred embodiment, the present invention uses paclitaxel as anticancer drug.

### Coating of Micro-Particle (I)

Micro-particle (I) in this invention can be coated with a biodegradable polymer to reduce the initial burst release of anticancer drug. The coating biodegradable polymer can be the same polymer used in micro-particle (I) or different polymer. Coating of micro-particle (I) can be done by various methods such as a coating method through the phase separation of coating polymer on the surface of PLGA micro-particles using emulsion-solvent evaporation method (H. Takabe et al. Pharmacology & Pharmacy, 578-583 (2014)) or a double-walled micro-particle fabrication method (Q. Xu et al. Biomaterials, 3902-3911 (2013)).

### Micro-Particle (II)

Micro-particle (II) consists of polyester and wound healing drugs and can be prepared with any one of the techniques described in the previous section. Micro-particle (II) degrades much faster than micro-particle (I) and releases wound healing drugs during the first 7-10 days. In a preferred embodiment, the present invention uses PLGA. Micro-particle (II) can be prepared with PLGA 1) PLA:PGA 50:50 2) molecular weight (Mw)<35,000 and 3) micro-particle size <50% of micro-particle (I) size. Preferably the size of micro-particle (II) is smaller than 50 μm. Typical composition of micro-particle (II) in this invention is 60-95% of PLGA and 5-40% of combined wound healing drugs. In one embodiment, micro-particle (II) contains borneol (monoterpene, 0.5-5%) and bismuth subgallate (1-10%) as wound healing drugs. Sulbogin® consisting of both drugs was approved by the U.S. FDA in 2004 as an ointment form for daily application and commercialized in the U.S. It is advantageous of using the drugs approved by the U.S. FDA. In a preferred embodiment, micro-particle (II) can be prepared with the same composition of borneol (0.7%) and bismuth subgallate (4.5%) used in Sulbogin®.

### Micro-Particle Delivery

Coated-micro-particle (I) and micro-particle (II) are mixed at a ratio of coated-micro-particle (I) to micro-particle (II) less than 0.5 and preferably 0.25 to make sure that the release amount of wound healing drugs surpasses that of anticancer drug during the first 7-10 days. The combined micro-particles (I) and (II) should be administered uniformly to the entire cancer removed area to avoid the overdose of some area. The amount of delivered micro-particles should be determined and adjusted depending on the size of the cancer removed area.

### Fabrication of Micro-Particle (I) by Microencapsulation Technique

Micro-particle (I) can be prepared by oil-in-water emulsion and solvent evaporation process. Briefly, paclitaxel (50 mg) is dissolved in a solution of PLGA (0.5 g; 75:25; Mw=˜400,000) in 5 mL of dichloromethane. The organic solution is added into 20 mL of 1% polyvinyl alcohol (PVA) aqueous solution. PVA is used to stabilize the emulsified micro-particles and prevent micro-particle fusion during the emulsion process. The resulting solution is rapidly homogenized using a homogenizer for 30 sec. The size of micro-particles can be controlled by the speed of the homogenizer. The mixture is then diluted with 0.1% PVA with a final volume of 500 mL, and stirred at 1000 rpm at room temperature and ambient pressure until the solvent evaporation is completed. The micro-particles are collected by centrifugation and washed with cold deionized water. The resulting micro-particles are then frozen using dry ice and dried in a lyophilizer.

### Coating of Micro-Particle (I)

The lyophilized micro-particle (I, 1 g) as prepared above is dispersed in 150 mL of a 0.1% PVA aqueous solution, and the resulting dispersion is added to 2 mL of a PLGA coating solution consisting of 100 mg of PLGA dissolved in 2 mL of acetone. The mixture is then stirred for 3 h at 40° C. The micro-particle (I) coated with PLGA is washed with cold deionized water and lyophilized. The lyophilized micro-particle (I) is stored at 4° C.

### Fabrication of Micro-Particle (II) by Co-Extrusion Technique

PLGA (50:50; Mw=˜45,000) in the form of granules is first milled at lower than 0° C. and sieved to obtain micro-particles having an average grain size of 180 μm or less. To this powder mass (10 g), is added finely pulverized borneol (70 mg; melting point=208° C.), bismuth subgallate (450 mg; melting point=223° C.) and other additives such as boric acid as a buffer and benzenesulfonamide (melting point=151° C.) as antimicrobial. The average grain size of two drugs and two additives is approximately 10 μm. The resulting mixture is homogenized in a mill at room temperature. The homogenized mixture is then extruded at 80-100° C. with a diameter of approximately 1.5 mm. The obtained rods are left to cool at room temperature, cut into small pieces and finally milled at −30° C. After sieving, micro-particle (II) with an average size of 25 μm or less but larger than 1 μm is collected. The resulting micro-particle (II) is stored at 4° C.

### Final Formulation

Micro-particle (I, 0.5 g) and micro-particle (II, 2 g) are mixed thoroughly and the resulting mixture is applied to the area from which the tumor was removed.

1. A method for treating a patient who recently was surgically treated for resection of a cancer mass, the method comprising:
administering a wound area that resulted from a cancer resection procedure with a plurality of micro-particles encompassing one or more cancer treatment drugs or medicaments;
a plurality of micro-particles encompassing one or more wound healing drugs or medicaments;
wherein said a plurality of micro-particles encompassing one or more cancer treatment drugs or medicaments are selectively released for a first period; and
wherein said a plurality of micro-particles encompassing one or more would healing drugs or medicaments are selectively released for a second period.

2. A method for treating a patient who recently was surgically treated for resection of a cancer mass as recited in claim 1, wherein said first period utilizes an increased physical size of the micro-particles to induce a reduced rate of release of one or more drugs or medicaments.

3. A method for treating a patient who recently was surgically treated for resection of a cancer mass as recited in claim 1, wherein said first period utilizes an increased molecular weight of the PLGA molecule to induce a reduced rate of release of one or more drugs or medicaments.

4. A method for treating a patient who recently was surgically treated for resection of a cancer mass, as recited in claim 1, wherein said first period utilizes an increased ratio of polylactic acid to polyglycolic acid in a PLGA molecule to induce a reduced rate of release of one or more drugs or medicaments.

5. A method for treating a patient who recently was surgically treated for resection of a cancer mass as recited in claim 1, where said first period utilizes one or more outer coatings on the micro-particle to induce a delayed release of one or more drugs or medicaments.

6. A method for treating a patient who recently was surgically treated for resection of a cancer mass as recited in claim 1, wherein said second period utilizes a decreased physical size of the micro-particles to induce an increased rate of release of one or more drugs or medicaments.

7. A method for treating a patient who recently was surgically treated for resection of a cancer mass as recited in claim 1, wherein said second period utilizes a decreased molecular weight of the PLGA molecule to induce an increased rate of release of one or more drugs or medicaments.

8. A method for treating a patient who recently was surgically treated for resection of a cancer mass, as recited in claim 1, wherein said second period utilizes a decreased ratio of polylactic acid to polyglycolic acid in a PLGA molecule to induce an increased rate of release of one or more drugs or medicaments.

9. A post-surgical treatment compound, said compound comprising:
a plurality of micro-particles encompassing one or more cancer treatment drugs or medicaments;
a plurality of micro-particles encompassing one or more wound healing drugs or medicaments;
wherein said plurality of micro-particles encompassing one or more cancer treatment drugs or medicaments has a first released period; and
where said a plurality of micro-particles encompassing one or more wound healing drugs or medicaments has a second released period.

10. A method for treating a patient who recently was surgically treated for resection of a cancer mass as recited in claim 9, wherein said first period utilizes an increased physical size of the micro-particles to induce a reduced rate of release of one or more drugs or medicaments.

11. A method for treating a patient who recently was surgically treated for resection of a cancer mass as recited in claim 9, wherein said first period utilizes an increased molecular weight of the PLGA molecule to induce a reduced rate of release of one or more drugs or medicaments.

12. A method for treating a patient who recently was surgically treated for resection of a cancer mass, as recited in claim 9, wherein said first period utilizes an increased ratio of polylactic acid to polyglycolic acid in a PLGA molecule to induce a reduced rate of release of one or more drugs or medicaments.

13. A method for treating a patient who recently was surgically treated for resection of a cancer mass as recited in claim 9, where said first period utilizes one or more outer coatings on the micro-particle to induce a delayed release of one or more drugs or medicaments.

14. A method for treating a patient who recently was surgically treated for resection of a cancer mass as recited in claim 9, wherein said second period utilizes a decreased physical size of the micro-particles to induce an increased rate of release of one or more drugs or medicaments.

15. A method for treating a patient who recently was surgically treated for resection of a cancer mass as recited in claim 9, wherein said second period utilizes a decreased molecular weight of the PLGA molecule to induce an increased rate of release of one or more drugs or medicaments.

16. A method for treating a patient who recently was surgically treated for resection of a cancer mass, as recited in claim 9, wherein said second period utilizes a decreased ratio of polylactic acid to polyglycolic acid in a PLGA molecule to induce an increased rate of release of one or more drugs or medicaments.
